(19) United States
(12) Patent Application Publication
(10) Pub. No.: US 2015/0343163 A1
(43) Pub. Date: Dec. 3, 2015

(54) BREATH SAMPLING TUBE AND DEVICE

(71) Applicant: ORIDION MEDICAL 1987 LTD., Jerusalem (IL)

(72) Inventor: David P. Besko, Thornton, CO (US)

(73) Assignee: ORIDION MEDICAL 1987 LTD., Jerusalem (IL)

(21) Appl. No.: 14/292,984

(22) Filed: Jun. 2, 2014

(51) Int. Cl. A61M 16/08; A61B 5/083; A61M 16/10; A61B 5/097; A61M 16/00; A61M 16/20

(52) U.S. Cl. CPC A61M 16/085; A61M 16/0003; A61M 16/20; A61M 16/1095; A61M 16/105; A61B 5/097; A61B 5/0836; A61M 2016/103; A61M 2205/3368; A61M 2205/75

(57) ABSTRACT

Breath sampling line including a sample cell housing and a breath sampling tube, the breath sampling tube having a first end connected to the sample cell housing, a second end configured to be connected to a respiratory output device, and a pervaporation tubing; the pervaporation tubing located at the first end of the breath sampling tube.

### TECHNICAL FIELD

The present disclosure generally relates to the field of breath sampling tubes and monitors.

### BACKGROUND

Accurate monitoring concentrations of gases, such as for example carbon dioxide (CO₂) in exhaled breath, is vital in assessing the physiologic status of a patient. Breath sampling is generally performed through breath sampling tubes configured to be connected to a patient airway and to a medical device.

Liquids are common in patient sampling systems, and have several origins, for example condensed out liquids from the highly humidified air provided to and exhaled from the patient. These liquids typically accumulate both in the patient airway and in the sampling line tubing; secretions from the patient, typically found in the patient airway; and medications or saline solution provided to the patient during lavage, suction and nebulization procedures.

### SUMMARY

The present disclosure relates to breath sampling lines configured to evaporate liquids.

One of the major obstacles when designing a sampling system is the necessity to prevent any liquids from reaching the sensitive and expensive components, such as sensors, while providing undisturbed sampling of the patient's breath at reduced costs.

Sampling lines are typically designed so that water vapor is wicked out and liquids absorbed prior to reaching the sensor/monitor. This typically requires both pervaporation tubings (at the patient end) and filters at the monitoring end of the sampling tube.

The breath sampling lines, disclosed herein, include breath sampling tubes and sample cell housings. The sample cell housings may include filter compartments containing therein a filter which may absorb condensed out liquids. The filter compartment may advantageously be rigid, thereby preventing moist air to circumvent the filter due to gaps formed between the breath sampling tube and the filter when bent.

Furthermore, the breath sampling tubes and/or medical devices disclosed herein may include a heating element configured to heat the pervaporation tubing of the medical device. The heating elements may be arranged such that when a breath sampling line is connected to the medical device, the heating element heats the pervaporation tubing of the breath sampling line, thereby increasing the efficiency of evaporation. In effect, the use of heating elements may advantageously obviate the need for filters in the sampling line, thereby reducing the cost of production of the sampling line, without jeopardizing the safety of sensitive parts of the monitoring equipment.

Certain embodiments of the present disclosure may include some, all, or none of the above advantages. One or more technical advantages may be readily apparent to those skilled in the art from the figures, descriptions and claims included herein. Moreover, while specific advantages have been enumerated above, various embodiments may include all, some or none of the enumerated advantages.

### BRIEF DESCRIPTION OF THE DRAWINGS

Examples illustrative of embodiments are described below with reference to figures attached hereto. In the figures, identical structures, elements or parts that appear in more than one figure are generally labeled with a same numeral in all the figures in which they appear. Alternatively, elements or parts that appear in more than one figure may be labeled with different numerals in the different figures in which they appear. Dimensions of components and features shown in the figures are generally chosen for convenience and clarity of presentation and are not necessarily shown in scale. The figures are listed below.

FIG. 1 schematically illustrates a prior art breath sampling line;

FIG. 2 schematically illustrates a breath sampling line, according to some embodiments;

FIG. 3 schematically illustrates a sample cell housing, according to some embodiments;

FIG. 4 schematically illustrates a sample cell housing, according to some embodiments;

FIG. 5 schematically illustrates a sample cell housing, according to some embodiments;

FIG. 6 schematically illustrates a breath sampling line including a heating element, according to some embodiments;

FIG. 7A schematically illustrates a medical device, including a heating element, before connection of a breath sampling tube, according to some embodiments;

FIG. 7B schematically illustrates a medical device, including a heating element, after connection of a breath sampling line thereto, according to some embodiments.

### DETAILED DESCRIPTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

According to some embodiments, there is provided a breath sampling line including a sample cell housing having a sample cell; and a breath sampling tube. The breath sampling tube may have a first end connected to the sample cell housing and a second end configured to be connected to a respiratory output device. The breath sampling tube may also include a pervaporation tubing located at the first end of the breath sampling tube, in proximity to the sample cell housing.

As used herein, the terms “breath sampling tube”, “sampling line” and “breath sampling line” may be interchangeably used and may refer to any type of tubing(s) or any part of tubing system adapted to allow the flow of sampled breath, for example, to an analyzer, such as a capnograph. The sampling line may include tubes of various diameters, adaptors, connectors, valves, and the like. Each possibility is a separate embodiment.

As used herein, the terms “respiratory output device” may refer to any type of respiratory device configured to be connected to the patient. Examples of suitable respiratory output devices may include oral and/or nasal cannulas, airway adaptors, or ventilation tubes. Each possibility is a separate embodiment. It is understood that other respiratory output devices enabling breath sampling may also be applicable and as such fall within the scope of the disclosure.

As used herein the term “pervaporation tubing” may refer to a tubing configured to wick out water vapor. For example water is absorbed by the pervaporation tubing whereafter it permeates the tubing until it reaches the outside wall of the tubing. Here it pervaporates into the surrounding gas (air or other gas). The permeability of the pervaporation tubing increases with temperature due to both decreased water viscosity and increased hydrophilic volume fraction.

According to some embodiments, the pervaporation tubing is a Nafion tube. Additionally or alternatively, the pervaporation tubing may be any kind of tubing configured to wick out water vapor, such as tubes including a hydrophilic material. According to some embodiments, the hydrophilic material may be a hydrophilic wicking material such as a porous plastic having a pore size ranging from approximately 5 microns to approximately 50 microns.

According to some embodiments, the term “sample cell housing”, as used herein, may refer to a hollow compartment configured to include therein the sample cell and optionally additional elements.

According to some embodiments, the term “sample cell” as used herein, may refer to a compartment within the housing in which the breath sample is analyzed.

According to some embodiments, the breath sampling tube may be devoid a filter. As used herein, the term “filter” may refer to membranes or other suitable elements, configured to absorb water and other liquids. According to some embodiments, the filter may include pores. According to some embodiments, the pores may let gases through, but are too small for liquids to pass through.

According to some embodiments, the breath sampling line may be disposable. Accordingly, breath sampling lines including breath sampling tubes devoid filters may be produced at significantly lower costs. This is further augmented by the fact that most breath sampling tubes have a very short life time and are disregarded prior to exhaustion of the relatively expensive filters, thereby causing unnecessary waste of resources and blown up expenses. It is understood by one of ordinary skill that such “low cost” sampling tubes require other means to wick out and/or absorb liquids, in order to compensate for the lack of filter. Features configured to compensate for low cost breath sampling tubes devoid of filters are disclosed herein.

According to some embodiments, the breath sampling tube may include a heating element configured to heat the pervaporation tubing. According to some embodiments, the heating element is positioned in proximity to the pervaporation tubing. As a non-limiting example, the heating element may include one or more LEDs; which, when turned on, may heat the pervaporation tubing. As another non-limiting example, the heating element may include a wire, such as a resistive wire. Exemplary resistive wires include alloys of FeCrAl, NiCr, NiFe, or CuNi, stainless steel, steels, aluminum, copper, or nickel. Each possibility is a separate embodiment. According to some embodiments, the wire may be wrapped around or otherwise attached to the tube. It is understood to one of ordinary skill in the art that the wire may be wrapped around the tube in a helical or non-helical manner. According to some embodiments, the heating element may include a conductive ink. According to some embodiments the conductive ink may be printed on the tube. According to some embodiments, the conductive ink may be printed on the tube in a circumferential manner.

It is understood by one of ordinary skill in the art that the breath sampling tube may include more than one heating element, such as but not limited to 2, 3, 4, 5, 10 or more heating elements. Each possibility is a separate embodiment.

According to some embodiments, the term “heating element” as used herein, may refer to any element configured to heat its proximate surroundings. According to some embodiments, the heating element is configured to heat the sampling tube (or parts thereof) to a temperature of approximately 37° C. According to some embodiments, the heating element is configured to heat the sampling tube (or parts thereof) to a temperature of above 37° C., such as in the range of approximately 37° C.-40° C. According to some embodiments, the heating element is configured to keep the temperature of the sampling tube (or part thereof) at a temperature of approximately 37° C. According to some embodiments, the heating element is configured to keep the temperature of the sampling tube (or part thereof) at a temperature of above 37° C., such as in the range of approximately 37° C.-40° C. It is understood to one of ordinary skill in the art that the dew point of the breath sample gas is 37° C., assuming the gas is 100% saturated. It is thus understood that heating the pervaporation tubing may contribute to the pervaporation efficiency of the pervaporation tubing. According to some embodiments, the heating element may be configured to pervaporate water vapor by conduction, convection, radiation or any combination thereof. Each possibility is a separate embodiment.

It is understood by one of ordinary skill in the art that keeping the breath sample, flowing in the pervaporation tubing, warm is conducive to the evaporation of water vapor. Hence, the breath sampling line, disclosed herein, including a heating element may enhance the evaporative properties of the pervaporation tubing. This enables placing the pervaporation tubing in proximity to the sample cell and may advantageously eliminate the need for an additional filter element along the breath sample tube. This is in contrast to conventional breath sampling tubes in which pervaporation tubings are connected to the sampling tube close to the patient (where the breath sample still retains body temperature) and which typically require an additional filter, in proximity to the sampling cell, in order to prevent that condensed out water, reaches sensitive parts of the sample cell.

According to some embodiments, the heating element may, additionally or alternatively, be configured to heat the sample cell. This may prevent water condensation in the sampling cell itself, which may be of particular importance during long measurement sessions.

According to some embodiments, the heating element may be configured to be turned on only when the breath sampling line is connected to a medical device. As a non-limiting example, the breath sampling line may include a conductive element arranged such that when the breath sampling line is connected to the medical device, an electrical circuit, including a power source and the heating element, is closed, thereby turning on the heating element. This may on the hand ensure that the heating element is not constantly turned on, causing unnecessary and environmentally harmful waste of energy, while on the other hand assuring that the breath sampling is always and automatically performed with the heating element turned on, such that liquids will not reach the sample cell. It is understood that the conductive element may be replaced by any suitable contact, switch or other element configured to trigger activation of the heating element, only when the breath sampling line is connected to the medical device.

According to some embodiments, the sample cell housing may include a thermally conductive material. Alternatively, the sample cell housing may be made of a thermally conductive material. Suitable thermally conductive materials include silver, copper, gold, carbon, nickel, tin, aluminum, molybdenum, zinc, lithium, tungsten, brass, iron, palladium, platinum, bronze, beryllium copper, phosphor bronze, iridium, magnesium, rhodium, silicon or combinations thereof. Each possibility is a separate embodiment. The thermally conductive material may contribute to the heating of the pervaporation tubing, provided by a heating element (whether positioned on the breath sampling line itself or in the medical device, as further described herein).

According to some embodiments, the sample cell housing may include a filter compartment. As used herein, the term “filter compartment” may refer to a chamber or a void in the housing configured to receive a filter. According to some embodiments, the filter compartment may be an integral part of the sample cell housing, thereby forming a one piece module therewith. According to some embodiments, making the filter part of the sample cell housing, rather than part of the breath sampling tube reduces the amount of tubing and glue steps when forming the a sampling line (such as a capnography sampling line). This since, when filters are incorporated into breath sampling tubes, a larger diameter tube, containing therein the filter, is required and consequently, the smaller diameter breath sampling tube must be glued (or otherwise attached) to the larger diameter tube containing the filter. A filter compartment, being an integral part of the sample cell housing, circumvents the need for large diameter tubes.

According to some embodiments, the filter compartment may be rigid. Being rigid may advantageously prevent that moist air will circumvent the filter due to gaps formed between the breath sampling tube and the filter when bent.

According to some embodiments, at least part of the filter compartment may be a molded on extension of the sample cell housing. Additionally or alternatively, at least part of the filter compartment may be located within the sample cell housing.

According to some embodiments, the filter compartment may be sealed off by the sample cell. For example, the sample cell window may be enlarged to extend beyond the sensing area in order for it to encapsulate the filter compartment. It is understood by one of ordinary skill in the art that it may only be necessary to extend one side of the sample cell to permit the installation of the filter. According to some embodiments, the filter compartment may be sealed off only when the filter is inserted into the filter compartment.

According to some embodiments, the heating element may be located on an external part of the breath sampling line. According to some embodiments, the heating element may be located in proximity to the pervaporation tubing. According to some embodiments, the heating element may be attached to, mounted on, embedded in or molded on or within the breath sampling line. Each possibility is a separate embodiment. According to some embodiments, the heating element may be an integral part of the breath sampling line.

According to some embodiments, there is provided a medical device comprising a CO₂ sensor and a heating element. The heating element may be arranged such that when a breath sampling line is connected to the medical device, the heating element heats at least part of the breath sampling line. According to some embodiments, the medical device is a capnographs. According to some embodiments, the CO₂ sensor is an infrared CO₂ sensor.

According to some embodiments, the heating element heats a pervaporation tubing of the breath sampling line. According to some embodiments, the pervaporation tubing is a Nafion tube. Additionally or alternatively, the pervaporation tubing may be any kind of tubing configured to wick out water vapor, such as tubes including a hydrophilic material. According to some embodiments, the hydrophilic material may be a hydrophilic wicking material such as a porous plastic having a pore size ranging from approximately 5 microns to approximately 50 microns.

According to some embodiments, the heating element is configured to heat the sampling tube (or parts thereof) to a temperature of approximately 37° C. According to some embodiments, the heating element is configured to heat the sampling tube (or parts thereof to a temperature of above 37° C., such as in the range of approximately 37° C.-40° C. According to some embodiments, the heating element is configured to keep the temperature of the sampling tube (or part thereof) at a temperature of approximately 37° C. According to some embodiments, the heating element is configured to keep the temperature of the sampling tube (or part thereof) at a temperature of above 37° C., such as in the range of approximately 37° C.-40° C. It is understood to one of ordinary skill in the art that the dew point of the breath sample gas is 37° C., assuming the gas is 100% saturated. It is thus understood that heating the pervaporation tubing may contribute to the pervaporation capability of the pervaporation tubing. According to some embodiments, the heating element may be configured to pervaporate water vapor by conduction, convection, radiation or any combination thereof. Each possibility is a separate embodiment.

It is understood by one of ordinary skill in the art that keeping the breath sample, flowing in the pervaporation tubing, warm is conducive to the evaporation of water vapor. Hence, the medical device, disclosed herein, including a heating element may enhance the evaporative properties of the pervaporation tubing. This enables placing the pervaporation tubing of the breath sampling tube in proximity to the sample cell and may advantageously eliminate the need for an additional filter element along the breath sample tube. According to some embodiments, the heating element may, additionally or alternatively, be configured to heat the sample cell. This may prevent water condensation in the sampling cell itself, which may be of particular importance during long measurement sessions.

According to some embodiments, the heating element may be configured to be turned on only when a breath sampling tube is connected to the medical device. As a non-limiting example, the medical device may include a switch arranged such that connecting a breath sampling tube to the sample housing may press upon the switch thereby turning on the heating element. It is understood that the switch may be replaced by any suitable contact or element configured to close an electrical circuit, and consequently supply power to the heating element, only when a breath sampling tube is connected to the sample cell housing. Alternatively, the medical device may include a light detector configured to detect light emitted from a light source positioned on the breath sampling tube, such that upon connection of a breath sampling tube to the medical device, a light signal is detected and activation of the heating element triggered. According to some embodiments, the light source of the breath sampling tube may be a LED. According to some embodiments, the same LED may also function as a heating element configured to heat the pervaporation tubing. It is understood by one of ordinary skill in the art that such triggered activation of the heating element may on the hand ensure that unnecessary and environmentally harmful waste of energy is avoided while on the other hand assuring that breath sampling is always and automatically performed with the heating element turned on, such that liquids will not reach sensitive parts of the medical device.

According to some embodiments, there is provided a breath sampling tube having a first end configured to be connected to a medical device and a second end configured to be connected to a respiratory output device. The breath sampling tube may include a pervaporation tubing located at the first end (device end) of the breath sampling tube. The breath sampling tube may also include a heating element. According to some embodiments, the heating element may be arranged such that when the tube is connected to a medical device, the heating element may heat the pervaporation tubing.

According to some embodiments, there is provided a breath sampling system including a medical device and a breath sampling line.

According to some embodiments, the medical device may include a CO₂ sensor. According to some embodiments, the medical device is a capnographs. According to some embodiments, the medical device may include a heating element. According to some embodiments, the heating element is arranged such that when the breath sampling line is connected to the sample cell housing, the heating element heats at least part of the breath sampling line.

According to some embodiments, the breath sampling line may include a sample cell housing and a breath sampling tube. According to some embodiments, the breath sampling tube has a first end connected to the sample cell housing and a second end configured to be connected to a respiratory output device. According to some embodiments, the breath sampling tube may also include a pervaporation tubing. The pervaporation tubing may be located at the first end of the breath sampling tube, in proximity to the sample cell housing.

As used herein, the term “at least a part of” may refer to the entire tube, the proximal end of the tube or the distal end of the tube. Each possibility is a separate embodiment.

As used herein, the terms “distal” and “distal end” may refer to the part of the tube closest to the subject. The length of the distal end may for example be 0.5, 1, 2, 3, 4, 5, 10 cm or more. Each possibility is a separate embodiment.

As used herein, the terms “proximal” and “proximal end” may refer to the part of the tube closest to the medical device. The length of the proximal end may for example be 0.5, 1, 2, 3, 4, 5, 10 cm or more. Each possibility is a separate embodiment.

As used herein, the term “proximity” may refer to 30, 20, 15, 10, 5, 1, 0.5 cm or less. Each possibility is a separate embodiment.

As used herein, the term “certain distance” may refer to a distance larger than 10 cm, for example larger than 20 cm, 30 cm, 40 cm or 50 cm, 70 cm. Each possibility is a separate embodiment.

As used herein, the term “at least a part of” may refer to the entire filter compartment, the proximal end of the filter compartment, or the distal end of the filter compartment. Each possibility is a separate embodiment.

As used herein, the terms “proximal” and “proximal end” may refer to the part of the filter compartment closest to the breath sampling tube. The length of the distal end may for example be 0.25, 0.5, 1, 2, 3, 4, 5 cm or more. Each possibility is a separate embodiment.

According to some embodiment, as used herein, the term “approximately”, with regards to a temperature, may refer to a temperature of ±0.5° C., relative to the temperature specified.

According to some embodiments, the breath sampling system may also include a first connector. According to some embodiments, the first connector may be configured to connect between the breath sampling tube and the sample cell housing. According to some embodiments, the breath sampling system may also include a second connector. According to some embodiments, the second connector may be configured to connect between the breath sampling tube and a patient respiratory output device. According to some embodiments, the connector is configured to connect to an oral/nasal cannula.

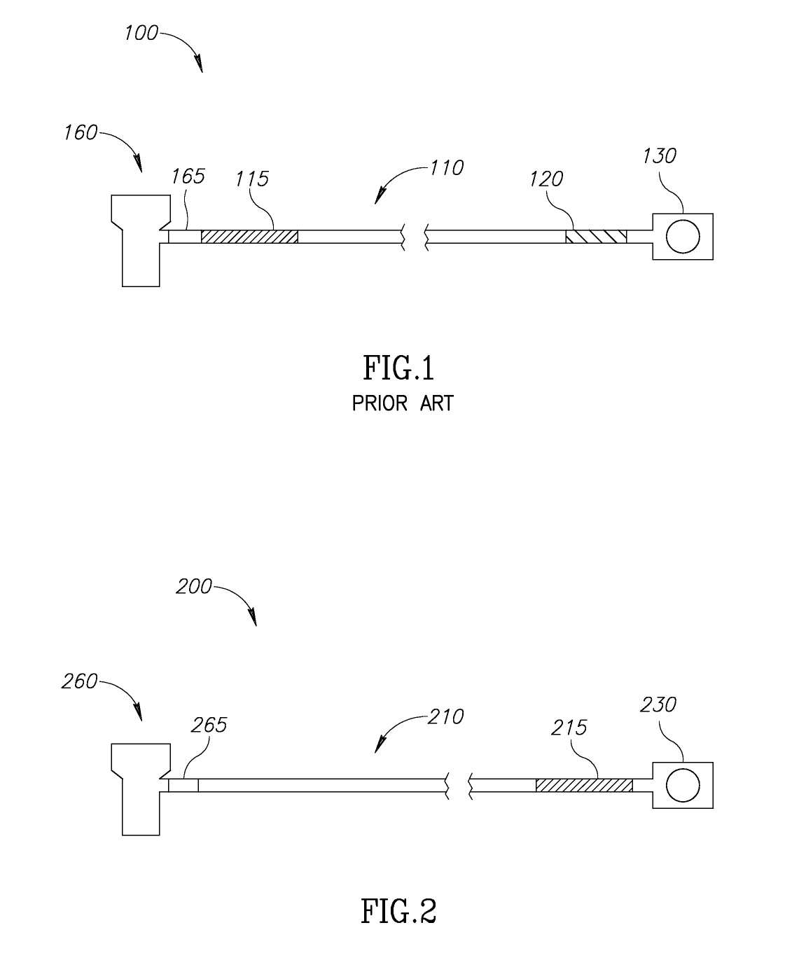

Reference is now made to FIG. 1, which schematically illustrates a prior art breath sampling line 100. Breath sampling line 100 includes a breath sampling tube 110 connected to a sample cell housing 130, at a first end thereof, and, through connector 165, to a respiratory output device 160 (here an airway adaptor), at a second end thereof. Breath sampling tube 110 includes a pervaporation tubing 115 (e.g. a Nafion tube). Pervaporation tubing 115 is configured to pervaporate water vapor and is most effective when kept warm. In effect, pervaporation tubing 115 is typically located at the second end of breath sampling tube 110 i.e. in proximity to respiratory output device 160 and hence the patient (not shown). However, as the breath sample proceeds through sampling tube 115, it cools down thereby causing remaining water vapor to be condensed out. Consequently, breath sampling tube 110 typically also includes a filter 120 configured to absorb water in its, prior to the breath sample reaching sample cell housing 130.

Reference is now made to FIG. 2, which schematically illustrates a breath sampling line 200, according to embodiments herein. Breath sampling line 200 includes a breath sampling tube 210 connected to a sample cell housing 230, at a first end thereof, and, through a connector 265, to a respiratory output device 260 (here an airway adaptor), at a second end thereof. Breath sampling line 200 is configured to be connected to a medical device (not shown) designed to enhance evaporation, as further disclosed herein. In effect, breath sampling tube 210 includes a pervaporation tubing 215 (e.g. a Nafion tube) located at the first end thereof, in proximity to sample cell housing 230. Advantageously, breath sampling tube 210 does not include a filter.

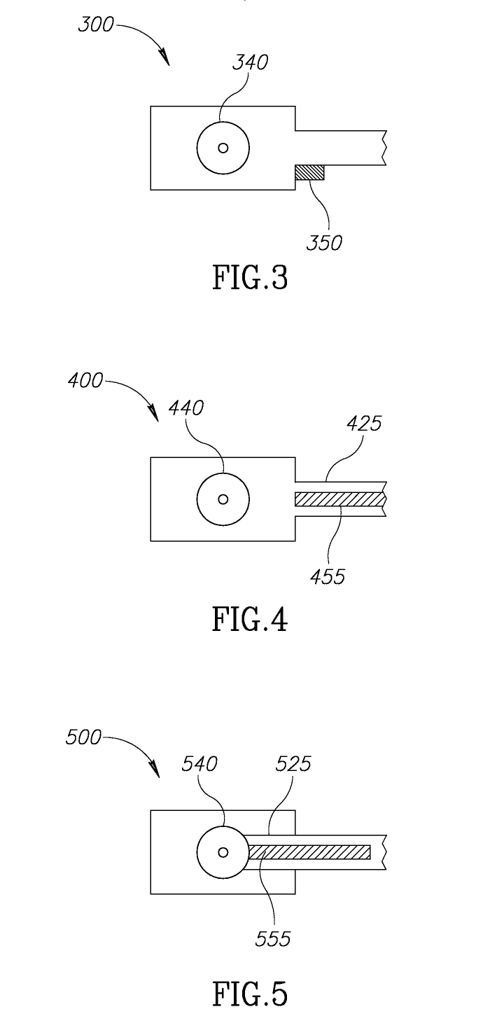

Reference is now made to FIG. 3, which schematically illustrates a sample cell housing 300, according to embodiments herein. Sample cell housing 300 includes therein a sample cell 340, configured to contain breath samples for analysis. Sample cell housing 300 is configured to be connected to a medical device (not shown) including a heating element 350 (as part of the medical device). Accordingly, when a pervaporation tubing (such as pervaporation tubing 215 of breath sampling tube 210) is connected to the medical device, heating element 350 heats pervaporation tubing 215, thereby ensuring effective pervaporation by pervaporation tubing 215. Heating element 350 may also be configured to heat sample cell 340, thereby ensuring that water vapor does not condense out within sample cell 340.

Reference is now made to FIG. 4, which schematically illustrates a sample cell housing 400, according to embodiments herein. Sample cell housing 400 includes therein a sample cell 440, configured to contain breath samples for analysis. Sample cell housing 400 further includes a filter compartment 425 configured to contain therein a filter 455. Filter compartment 425 is formed as a molded on extension of sample cell housing 400, thereby forming a one-piece module therewith. Accordingly, filter 455 absorbs condensed out liquids from the breath sampling tube (such as breath sampling tube 210) prior to the liquids reaching sample cell 440.

Reference is now made to FIG. 5, which schematically illustrates a sample cell housing 500, according to embodiments herein. Sample cell housing 500 includes therein a sample cell 540, containing breath samples for analysis. Sample cell housing 500 further includes a filter compartment 525 containing therein a filter 555. Filter compartment 525 is formed as a compartment within sample cell housing 500, thereby forming a one-piece module therewith. Accordingly, filter 555 absorbs condensed out liquids from the breath sampling tube (such as breath sampling tube 210) connected thereto, prior to the liquids reaching sample cell 540. Filter compartment 525, and thus filter 555, may be sealed off by sample cell 540, for example by enlarging sample cell 540 to extend beyond the area of sampling.

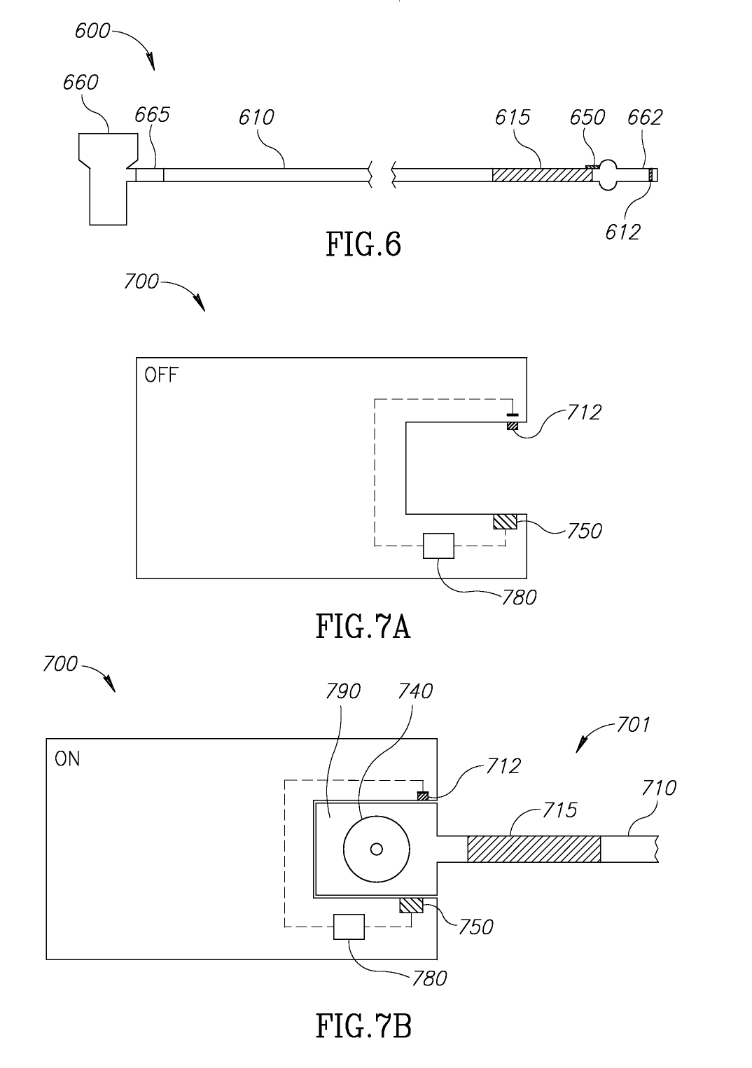

Reference is now made to FIG. 6, which schematically illustrates a breath sampling line 600, according to embodiments herein. Breath sampling line 600 is configured to be connected, through a first connector 662, to a medical device (not shown) at a first end thereof and, through a second connector 665, to a respiratory output device 660 (here an airway adaptor), at a second end thereof. Breath sampling line 600 includes a pervaporation tubing 615 at the first end thereof, in proximity to the medical device, and a breath sampling tube 610 at the second end thereof, in proximity to respiratory output device 660. Breath sampling line 600 also includes a heating element 650 at the first end thereof. Accordingly, when pervaporation tubing 615 of breath sampling line 600 is connected to the medical device, heating element 650 heats pervaporation tubing 615, thereby ensuring effective pervaporation by pervaporation tubing 615. In effect, pervaporation tubing 615 may be assembled with breath sampling tube 610 in proximity to the medical device rather than in proximity to the patient, as typically required. Likewise, breath sampling line 600 does not require additional filters, as heating element 650 ensures efficient pervaporation of water by pervaporation tubing 615. Breath sampling line 600 also includes a conductive element 612 arranged such that when breath sampling tube 600 is connected to the medical device, an electrical circuit (including a power source (not shown) and heating element 650), is closed, thereby turning on heating element 650.

Reference is now made to FIGS. 7A and 7B, which schematically illustrate a medical device 700, including a heating element 750, according to some embodiments, before and after connection of a sampling line 701. Sampling line 701 includes a pervaporation tubing 715, a breath sampling tube 710 and a sampling cell housing 790 having a sampling cell 740, as described herein. Heating element 750 is configured to heat pervaporation tubing 715 when sampling tube 710 is connected to medical device 700 (as in FIG. 7B). Medical device 700 may further include a power source 780 electrically connected to heating element 750 and a switch 712 configured to close an electrical circuit including heating element 750, and consequently supply power thereto, when breath sampling tube 710 is connected to medical device 700 (as illustrated in FIG. 7B). When no tube is connected to medical device 700, the electrical circuit remains open and heating element 750 remains deactivated (as in FIG. 7A).

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” or “comprising,” when used in this specification, specify the presence of stated features, integers, steps, operations, elements, or components, but do not preclude or rule out the presence or addition of one or more other features, integers, steps, operations, elements, components, or groups thereof.

While a number of exemplary aspects and embodiments have been discussed above, those of skill in the art will recognize certain modifications, additions and sub-combinations thereof. It is therefore intended that the following appended claims and claims hereafter introduced be interpreted to include all such modifications, additions and sub-combinations as are within their true spirit and scope.

1. A breath sampling line comprising:
a sample cell housing comprising a sample cell;
and a breath sampling tube, said breath sampling tube comprising a first end connected to said sample cell housing, a second end configured to be connected to a respiratory output device, and a pervaporation tubing; said pervaporation tubing located at said first end of said breath sampling tube.

2. The breath sampling line of claim 1, wherein said breath sampling tube is devoid a filter.

3. The breath sampling line of claim 1, wherein said sample cell housing comprises a thermally conductive material.

4. The breath sampling line of claim 1, further comprising a heating element configured to heat said pervaporation tubing.

5. The breath sampling line of claim 4, wherein said heating element is configured to heat said pervaporation tubing to a temperature of approximately 37° C.

6. The breath sampling line of claim 4, wherein said heating element is configured to keep the temperature of said pervaporation tubing at a temperature of approximately 37° C.

7. The breath sampling line of claim 4, wherein said heating element is configured to be turned on only when said breath sampling line is connected to a medical device.

8. The breath sampling line of claim 4, further comprising a conductive element arranged such that when said breath sampling line is connected to a medical device, said conductive element closes an electrical circuit thereby activating said heating element.

9. The breath sampling line of claim 1, wherein said sample cell housing further comprises a filter compartment configured to receive a filter therein.

10. The breath sampling line of claim 9, wherein said filter compartment is rigid.

11. The breath sampling line of claim 9, wherein at least part of said filter compartment is a molded on extension of said sample cell housing.

12. The breath sampling line of claim 9, wherein at least part of said filter compartment is located within said sample cell housing.

13. The breath sampling line of claim 12, wherein said filter compartment is sealed off by said sample cell.

14. A medical device comprising a CO₂ sensor and a heating element; said heating element arranged such that when a breath sampling line is connected to said medical device, said heating element heats at least part of said breath sampling line.

15. The medical device of 14, wherein said heating element heats a pervaporation tubing of said breath sampling line.

16. The medical device of claim 15, wherein said heating element is configured to heat said pervaporation tubing to a temperature of approximately 37° C.

17. The medical device of claim 16, wherein said heating element is configured to keep the temperature of said pervaporation tubing at a temperature of approximately 37° C.

18. The medical device of claim 14, wherein said heating element is configured to be turned on only when said breath sampling tube is connected to said sample cell housing.

19. The medical device of claim 18, further comprising a switch arranged such that when said breath sampling tube is connected to said medical device, said switch triggers activation of said heating element.

20. A breath sampling tube comprising a first end configured to be connected to a medical device, a second end configured to be connected to a respiratory output device, a pervaporation tubing, and a heating element; wherein said pervaporation tubing is located at said first end of said breath sampling tube.
